(19) United States
(12) Patent Application Publication
(10) Pub. No.: US 2025/0152503 A1
(43) Pub. Date: May 15, 2025

(54) IMPLANTABLE DEVICES FOR THE SUSTAINED DELIVERY OF AN OPIOID ANTAGONIST AND METHODS FOR TREATING INFLAMMATORY, NEUROINFLAMMATORY AND METABOLIC DISORDERS

(71) Applicant: Delpor, Inc., Brisbane, CA (US)

(72) Inventor: Gregory A. Watkins, Oakland, CA (US)

(21) Appl. No.: 18/840,456

(22) PCT Filed: Feb. 21, 2023
(86) PCT No.: PCT/US2023/062962

§ 371 Date: Aug. 21, 2024

Related U.S. Application Data
(60) Provisional application No. 63/312,792, filed on Feb. 22, 2022.

(51) Int. Cl. A61K 9/00; A61K 9/20; A61K 31/485

(52) U.S. Cl. CPC A61K 9/0024; A61K 9/2013; A61K 9/2027; A61K 9/2095; A61K 31/485

(57) ABSTRACT

Diffusion-based, implantable devices for the delivery of an opioid antagonist, such as naltrexone, are described. The devices are cylindrical or discoidal in shape and fitted with a biocompatible membrane. These are filled with a compressed formulation of the opioid antagonist, and optionally, a binder. Upon introduction of an aqueous fluid into the device, the compressed formulation erodes at a constant rate along one axis to release the opioid antagonist from the device at a constant rate. The rate is selected to range from approximately 0.02-2.00 mg/day for the treatment of inflammatory, neuroinflammatory, or metabolic disorders.

### TECHNICAL FIELD

The subject matter described herein relates to implantable devices that comprise an opioid antagonist composition suitable to treat inflammatory, neuroinflammatory, and metabolic disorders.

### BACKGROUND

Opioid antagonists comprise a class of drugs including naltrexone, naloxone, and nalmefene that have been widely used since the 1970s to treat opioid use disorder (OUD) and alcoholism. Most of these drugs are potent, but rapidly metabolized and cleared following oral administration. For instance, when administered as a supportive treatment for opioid use disorder, a typical oral dose of naltrexone is 50 mg/day, with an average bioavailability of 5-40% and a plasma half-life of approximately 4 hours. This pulsatile method of dosing does not maintain a constant plasma level of the drug, but it does maintain a high degree of μ- and κ-opioid receptor occupancy over time. Consequently, such a dose is suitable to protect a patient from the euphoric effects of opioid receptor agonists such as morphine, heroin, and oxycodone.

Although naltrexone, naloxone, nalmefene, and similar drugs were developed as antidotes and prophylactic agents to treat opioid overdose and opioid use disorder, some recent research suggests that they may also be used to treat a wide range of inflammatory, neuroinflammatory, gastrointestinal, and metabolic disorders. These include psoriasis, fibromyalgia, HIV-associated neurological and neurocognitive disorders, Crohn's disease, multiple sclerosis, and cachexia. It has been postulated that naltrexone-like opioid antagonists act in these cases as immunomodulators—specifically, by antagonizing Toll-like receptor 4 (TLR4) signaling, thereby reducing the downstream production of cytokines and pro-inflammatory agents such as IL-1, TNF-α, and interferon-β. Conversely, long-term activation of TLR4—for instance, by long-term use of conventional opioids such as morphine, or by low levels of HIV trapped in CNS reservoirs—has been associated with the emergence of neuropathic pain and cognitive deficits.

TLR4 is expressed in numerous cell and tissue types, including microglial cells in the CNS, epithelial cells in the gastrointestinal tract, and in a wide variety of cancers. TLR4 overexpression is considered to be both pro-inflammatory and pro-carcinogenic, and expression levels positively correlate with proliferation rate in neoplastic cell lines. For instance, a well-characterized breast cancer cell line (MDA-MB-231) that expresses high basal levels of TLR4 was found to become less invasive following TLR4 knockdown.

Although not originally developed as anti-inflammatory agents, opioid antagonists and closely related molecules have been found to antagonize TLR4 signaling, including (+)-naltrexone, (−)-naltrexone, (+)-naloxone, (−)-naloxone, and (−)-nalmefene. There is also clinical evidence that therapeutically useful levels of TLR4 binding by the levorotatory isomers of naltrexone, naloxone, and nalmefene can be obtained with oral doses much smaller than those required to blockade μ- and κ-opioid receptors in OUD patients. For instance, “Low Dose Naltrexone” (LDN) therapy refers to the oral administration of naltrexone (generally, (−)-naltrexone) at doses of approximately 0.5-5 mg/day to treat a range of inflammatory disorders. This corresponds to approximately 1-10% of the oral dose typically administered to treat OUD. However, as noted above, naltrexone is rapidly metabolized and cleared following oral administration; hence, a large fraction of any oral dose is “wasted,” in that plasma levels must spike far above the minimum effective plasma concentration in order to sustain a therapeutic effect throughout a 24-hour plasma clearance period (equivalent to ˜6 half-lives of naltrexone).

Many dosage forms of naltrexone, naloxone, and similar drugs exist, but most are not ideal for treating inflammatory or metabolic disorders at low drug concentrations. For instance, (−)-naltrexone is typically provided to patients in the form of tablets (50 mg of the hydrochloride salt, dosed daily; REVIA®) or a depot injection (380 mg of the base, dosed monthly; VIVITROL®). Dosage forms currently under development include the O'Neil Long-Acting Naltrexone Implant (OLANI), a subcutaneous implant consisting of erodible pellets (1.8 g total mass, designed to deliver approximately 1.0 g. of naltrexone over a 6 month treatment period). These dosage forms were explicitly developed to treat alcoholism and OUD, and typically produce very high and uneven plasma levels over time. Additionally, in the case of OLANI, the large mass of naltrexone required to treat a patient for OUD for a 6 month period contributes to the large size of the implant and the invasive nature of the implantation procedure.

Although many factors impact the release kinetics of tablets and erodible depot drug delivery systems, one important factor is likely related to the change in physical surface area of the dosage form over time. Generally speaking, if a non-porous object is immersed in a volume of fluid and allowed to dissolve, the surface area of the solid object should gradually decrease. Furthermore, if one assumes that the fluid exterior to the object cannot be saturated (for instance, if the fluid volume is large, or if the fluid is part of an open system) and if the absolute dissolution rate of the object is roughly proportional to its surface area, then its instantaneous rate of dissolution should also decrease over time. If the object in question is an erodible pellet or mass containing a dispersed drug, and the fluid is a buffered biological fluid such as interstitial fluid, then the rate at which the drug is delivered into its environment should also decrease over time.

In summary, naltrexone and related opoid antagonists show promise as repurposed drugs to treat inflammatory and metabolic disorders, but existing commercial dose forms are suboptimal for these applications. Because these drugs were originally designed to treat alcoholism and OUD, existing oral and depot formulations are tailored to deliver relatively high doses of these drugs in a pulsatile or uneven fashion over time. Patients seeking relief from chronic inflammatory and metabolic disorders could benefit from much lower daily dose rates of drug over prolonged periods of time. Additionally, patients suffering from comorbidities of an inflammatory nature and/or a high pill burden (e.g., late-stage AIDS patients or those with cachexia) could benefit from extremely long-acting anti-inflammatory agents that do not require frequent dosing. One solution could take the form of a subcutaneous, non-erodible implant that releases an opioid antagonist (e.g., naltrexone) from an internal reservoir by controlled, slow dissolution of a solid formulation.

By way of illustration, in one version of such a device, a hollow cylinder or disc is tightly packed with a solid formulation of an opioid antagonist drug, such as (+)-naltrexone, (−)-naltrexone, (+)-naloxone, (−)-naloxone, (+)-nalmefene, (−)-nalmefene, and sealed at one or both circular ends with a membrane. The formulation may comprise entirely, or in part, either 1.) the free base form of an opioid antagonist such as naltrexone, or 2.) a salt form of an opioid antagonist such as naltrexone, formed by reacting the drug (as a base) with a pharmaceutically acceptable acid. Upon wetting of the membrane and the formulation, the housing of the reservoir restricts diffusion in two dimensions (i.e., where the housing contacts the formulation and restricts intrusion of the fluid), while the membrane(s) facilitate fluid contact and drug diffusion along a third dimension. Stated another way, the solid preferentially dissolves at a constant rate along a single axis corresponding to the height of the cylinder or thickness of the disc, instead of isotropically. In such a scenario, drug release kinetics may approach zero order. In other cases, the output from the device can be limited by the surface area and porosity of the membrane surfaces rather than the dissolution rate of the formulation. For instance, a cylindrical implantable device may have a small diameter on the order of 1-5 mm to minimize discomfort to the patient and limit the membrane surface area such that the rate of drug diffusion through the circular membrane is less than the rate at which the drug formulation dissolves within the device. Additional modulation of the output rate may be made by placing membranes at both ends of the device (to increase output) or a single end of the device (to decrease output). The target output rate (expressed, for instance, in mg of drug/day/device) must provide sufficient patient exposure after implantation as to provide the therapeutic effect (e.g., a reduction of inflammation) throughout a prolonged treatment period (e.g., 1-24 months). At the same time, there must be a sufficient mass of drug loaded within a device to support its target output rate and operative period, and yet the device cannot be too large as to become difficult to implant or remove, or create discomfort for the wearer. Questions of output rate and device size are complicated by the limited aqueous solubility of many opioid drugs in aqueous fluids; depending on the device design, the pure drug may or may not dissolve at a sufficiently high rate to drive diffusion across one or more membranes with a limited surface area. For instance, naltrexone has a reported aqueous solubility of 667 mg/L at 25° C.; this is substantially higher than the reported solubilities of nalmefene and buprenorphine, two other drugs used to treat opioid use disorder (values of 140 mg/L and 168 mg/L, respectively). To obtain an adequate diffusion rate from an implant built using these less soluble drugs, it might be necessary to either 1.) increase the membrane surface area of the device by increasing its volume or changing its shape, or 2.) add excipients that enhance the solubility of the drug within the device, but at the expense of decreasing the drug load or (again) increasing the overall device volume. There is also the question of whether the added excipient would remain stable over time within the device. For instance, many salt forms of opioid antagonists have greater solubility in water than the free base form of the drug; e.g., naltrexone hydrochloride has an estimated water solubility of 100 g/L at 25° C. Aside from the fact that such a salt may dissolve too rapidly for sustained release applications, these salt forms are also prone to hydrolysis and other forms of chemical degradation, and the rate of drug decomposition can depend on other features of the device, such as the rate at which physiological buffering species diffuse into a device from its operating environment. These factors illustrate the complexity of pairing a drug composition to a specific device design, particularly one that relies upon diffusion to release the drug from an internal reservoir. Formulation-device pairings that address these, and other, complications related to sustained and controlled delivery of opioid antagonists for anti-inflammatory and immunomodulatory applications are needed.

### BRIEF SUMMARY

The following aspects and embodiments thereof described and illustrated below are meant to be exemplary and illustrative, not limiting in scope.

In one aspect, a device comprising a composition comprised of an opioid antagonist is provided. The device has an elongated shape with an interior reservoir, and it has first and second ends. A membrane is positioned at one or both ends. In one embodiment, the device is dimensioned to have a length of less than or equal to about 8 cm and an inner reservoir diameter of about 8 mm. In another embodiment, the device has dimensions of less than or equal to about 5 cm in length and an inner reservoir diameter of about 5 mm.

The walls of the provided device are fashioned from a biocompatible, non-erodible material such as stainless steel or titanium.

The interior reservoir of the device contains, in one embodiment, the composition comprised of an opioid antagonist in the form of a compacted solid comprising, consisting essentially of, or consisting of an opioid antagonist, preferably in a solid or a compacted solid form. In another embodiment, the composition is comprised of an opioid antagonist as a suspension or solution or a solid. In yet another embodiment, the opioid antagonist is prepared as a solid solution or dispersion within a wettable solid matrix.

In one embodiment, the membrane at one or both ends of the device are circular. In one embodiment, the device has a cylindrical shape.

In one embodiment, the opioid antagonist is naltrexone base.

In another embodiment, the opioid antagonist is a stereoisomer or racemic mixture of naloxone, nalmefene, nalorphine, nalodeine, levallorphan, xorphanol, oxilorphan, samidorphan, 6-beta-naltrexol, methylnaltrexone, diprenorphine or cyprodime.

In one embodiment, the composition comprising the opioid antagonist is fabricated into a shape, such as a tablet, pellet or a rod, sized for placement in the interior reservoir of the device. In one embodiment, the shape is a compressed shape.

In another embodiment, the composition of opioid antagonist is fabricated into a pellet or rod having an outer diameter that greater than or equal to about 95%, 96%, 97%, 98% or 99% or 99.5% of the inner diameter of the device's interior reservoir.

In yet another embodiment, the composition comprising the opioid antagonist further comprises a binder. Exemplary binders include lactose, maltose, hydroxypropylcellulose, polyvinylpyrrolidone, gelatin, dextran, alginate, maltodextrin and polyethyleneglycol. In one embodiment, the binder facilitates fabrication of the composition into a compressed shape, such as a tablet, pellet or rod. In one embodiment, the compressed shape has a diameter greater than or equal to about 80%, 85%, 90%, 95%, 96%, 97%, 98% or 99% or 99.5% of the inner diameter of the device's interior reservoir.

In one embodiment, the composition comprising the opioid antagonist excludes an organic acid. In an embodiment, the organic acid (i) has a water solubility at room temperature of less than about 20 g/L, (ii) maintains a pH of the suspension in its environment of use of between 3-6.5 for a period of at least about 30 days, and/or (iii) has a molecular weight less than or equal to 500 grams per mole. That is, in an embodiment, the composition comprising the opioid antagonist excludes an organic acid that (i) has a water solubility at room temperature of less than about 20 g/L, (ii) maintains a pH of the suspension in its environment of use of between 3-6.5 for a period of at least about 30 days, and (iii) has a molecular weight less than or equal to 500 grams per mole.

In yet another embodiment, the composition comprising the opioid antagonist is prepared such that the opioid antagonist is frozen within a solid solution or dispersed within a wettable solid matrix. The solid matrix has a melting point greater or equal to about 40° C. Suitable matrix materials include polyvinylpyrrolidone, polyethylene glycol, methylcellulose, a fatty acid or fatty alcohol containing a minimum of 12 carbon atoms, a dicarboxylic acid containing a minimum of 8 carbons, or any mixture thereof. The solid matrix may be cast or fabricated into a compressed or extruded shape, such as a tablet, pellet, or rod, with a diameter greater than or equal to about 80%, 85%, 90%, 95%, 96%, 97%, 98%, 99%, or 99.5% of the interior diameter of the device.

In one embodiment, the composition comprising the opioid antagonist includes one or more stabilizing excipients. Such excipients include metal chelating agents, such as a salt of ethylenediaminetetraacetic acid; a buffering agent, such as a phosphate salt, a carboxylate salt, or an amino acid; and antioxidants, such as ascorbic acid, an ascorbic acid ester, tocopherol, a tocopherol ester, cysteine, a cysteine ester or amide, methionine, a methionine ester or amide, butylated hydroxytoluene (BHT), or butylated hydroxyanisole (BHA).

In another embodiment, the composition comprising the opioid antagonist comprises a constituent for dissolution of the composition. In one embodiment, the constituent is a sugar, such as sucrose or mannitol. In another embodiment, the composition comprising a constituent for dissolution of the composition is in the form of a solid, compacted pellet or rod or a compressed powder. In another embodiment, the composition is formulated to facilitate dissolution of the solid, compacted tablet, pellet or rod or compressed solid shape when hydrated within the interior reservoir of the device into an aqueous solution. In one embodiment, the composition is formulated to be a lyophilized powder. In one embodiment, the composition is a lyophilized composition comprised of the opioid antagonist and a sugar.

In one embodiment, the device is provided with the composition in the interior reservoir in a dry state. In another embodiment, the device is provided with the composition in the interior reservoir in a hydrated state. In yet another embodiment, the device is provided with the composition in the interior reservoir as a dry, compressed solid, and the compressed solid is wetted or moistened before or during use by an introduced aqueous fluid into which the opioid antagonist dissolves, partially or completely. For example, subsequent to implanting the device in a subject, body fluid will enter the device to create an aqueous phase within the interior reservoir and the aqueous phase comprises dissolved opioid antagonist. The aqueous phase may also contain undissolved opioid antagonist. Exemplary aqueous fluids include biocompatible fluids, such as sterile water, 0.9% saline, and phosphate buffered saline. In one embodiment, the compressed solid is shaped to conform to the dimensions of the interior reservoir of the device. In one embodiment, the compressed solid has a shape that is a tablet, a rod or a pellet.

In another embodiment, the device has a geometry that is cylindrical or discoidal.

In yet another embodiment, the device is configured for subcutaneous implantation into a human or other vertebrate animal.

In another embodiment, the device contains either a single circular membrane, or two circular membranes mounted in parallel at the ends of the device. The total diffusive area of the membrane(s) is preferably between about 1 mm² and 50 mm².

In another embodiment, the device membranes are selected from an inert fluoropolymer, such as polyvinylidine difluoride (PVDF), a sintered metal, such as a stainless steel or titanium metal, or a ceramic, such as alumina, titania, or silica.

In yet another embodiment, the device is loaded with a sufficient quantity of the opioid antagonist composition to provide a selected plasma level of the opioid antagonist for a therapeutic effect for an operative period of greater than or equal to about 1 month. In one embodiment, the device comprises a composition comprised of naltrexone in an amount generate an average plasma level of between about 0.01-0.50 ng/mL, between about 0.05-0.5 ng/mL, between about 0.08-0.5 ng/mL, or between about 0.1-0.5 ng/ml of naltrexone active moiety (defined as naltrexone plus 6-β-naltrexol, its active metabolite), following subcutaneous implantation in a human subject for an operative period of greater than or equal to about one month. In one embodiment, the device comprises a composition comprised of naltrexone in an amount generate an average plasma level of at least about 0.01 ng/ml, 0.02 ng/mL, 0.03 ng/ml, 0.04 ng/ml, 0.05 ng/mL, 0.06 ng/ml, 0.07 ng/mL, 0.08 ng/mL, 0.09 ng/ml or 0.1 ng/ml and less than 0.50 ng/ml of naltrexone active moiety (defined as naltrexone plus 6-β-naltrexol, its active metabolite), following subcutaneous implantation in a human subject for an operative period of greater than or equal to about one month.

In another embodiment, the composition in the interior reservoir of the device hydrates in the presence of a sterile or filtered aqueous solution to form an aqueous phase within the device interior reservoir that contains at least some of the opioid antagonist in a dissolved form. In another embodiment, the composition in the interior reservoir of the device hydrates in the presence of a sterile or filtered aqueous solution to form an aqueous phase within the device interior reservoir that contains at least a portion of the opioid antagonist in a suspended form.

In another embodiment, the opioid antagonist is released from the device at a rate that provides a therapeutic effect for the treatment period.

In another embodiment, multiple devices, preferably 1-6, 1-5,1-4, 2-6, 2-5, 2-4, or 2-3 devices, are administered to the subject. In one embodiment, multiple devices are administered to achieve a target opioid antagonist concentration in plasma, such as a naltrexone active moiety concentration of 0.01-0.50 ng/mL, between about 0.05-0.5 ng/mL, between about 0.08-0.5 ng/mL, or between about 0.1-0.5 ng/mL. In one embodiment, multiple devices are administered to achieve a target opioid antagonist concentration in plasma, such as a naltrexone active moiety concentration of at least about 0.01 ng/mL, 0.02 ng/mL, 0.03 ng/ml, 0.04 ng/mL, 0.05 ng/mL, 0.06 ng/mL, 0.07 ng/mL, 0.08 ng/mL, 0.09 ng/ml or 0.1 ng/ml and less than 0.50 ng/mL. In one embodiment, the device(s) is/are administered by implantation, and in another embodiment, the device(s) is/are implanted subcutaneously.

In another aspect, a method for sustained, controlled delivery of a small molecule opioid antagonist is provided, where the method comprises providing a device comprising a composition as described herein. In some embodiments, the method further comprises administering the device, such as by subcutaneous implantation.

In embodiments, a method for treating a disorder selected from psoriasis, fibromyalgia, HIV-associated neurological and neurocognitive disorders, Crohn's disease, multiple sclerosis, Complex Regional Pain Syndrome, Hailey-Hailey Disease, insulin resistance, pain or cachexia is provided.

In another aspect, a method to provide maintenance therapy to treat a chronic inflammatory, neuroinflammatory, or metabolic disorder is provided, where the method comprises providing a device comprising a composition as described herein. In some embodiments, the method further comprises administering the device, such as by subcutaneous implantation.

In addition to the exemplary aspects and embodiments described above, further aspects and embodiments will become apparent by reference to the drawings and by study of the following descriptions.

Additional embodiments of the present methods, devices and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIGS. 1A-1B are illustrations of a drug delivery device, in assembled form (FIG. 1A) and in unassembled form (FIG. 1B).

FIGS. 1C-IF illustrate a portion of a first exemplary drug delivery device, showing the end cap subassembly in cross section when fully assembled (FIG. 1C) and in an exploded view (FIG. 1D), and in isometric view when assembled (FIG. 1E). FIG. 1F shows an exploded view of the cap subassembly alone. The numbered elements of the subassembly are 1=cap, 2=porous membrane, 3=seal, 4-retention ring, and 5=drug device reservoir.

FIGS. 1G-1K illustrate a portion of a second exemplary drug delivery device, showing the end cap subassembly in cross section when fully assembled (FIG. 1G) and in an exploded view (FIG. 1H), and in isometric view when assembled (FIG. 1I). FIGS. 1J-1K show an assembled and exploded view of the cap subassembly alone. The numbered elements of the subassembly are 1=cap, 2=porous membrane, 3=seal, 4-drug delivery device reservoir, and 5=retention ring.

FIG. 2 is a graph showing the cumulative in vitro release of naltrexone base, in mg, as a function of time, in days, from two sets of devices (n=3 per set) comprising a composition of naltrexone base. Devices in the first set were filled with approximately 325 mg of naltrexone base in the form of a fine powder. Devices in the second set were filled with approximately 570 mg of tablets consisting of naltrexone base combined with 5% polyvinylpyrrolidone (40 kD) and 2% stearic acid.

FIG. 3 is a graph showing the naltrexone plasma levels, in ng/mL, as a function of time, in days, in rats implanted with singular devices comprising a composition of naltrexone base. One set of rats (n=5) received devices loaded with approximately 325 mg of finely powdered naltrexone base. A second set of rats (n=3) received devices loaded with approximately 570 mg of tablets consisting of naltrexone base combined with 5% polyvinylpyrrolidone (40 kD) and 2% stearic acid by mass.

### DETAILED DESCRIPTION

### Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about,” when immediately preceding a numerical value, means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts and ratios are based upon the total weight of the compositions and all measurements made are at about 25° C., unless otherwise specified.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The term “treating” is used herein in reference to methods of administration of a small molecule which reduces the intensity or frequency of, or delays the onset of, symptoms of a medical condition (e.g., an inflammatory, neuroinflammatory, or metabolic disorder such as psoriasis, fibromyalgia, HIV-associated neurological and neurocognitive disorders, Crohn's disease, multiple sclerosis, Complex Regional Pain Syndrome, Hailey-Hailey Disease, insulin resistance, pain or cachexia) in a subject relative to a subject not receiving the compound or composition. This can include reversing, reducing, or arresting the symptoms, clinical signs, and underlying pathology of a condition in a manner to improve or stabilize a subject's condition.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### Device and Composition

In one exemplary embodiment, a device comprises a composition (or formulation) that comprises an opioid antagonist, such as naltrexone base or a pharmaceutically acceptable salt form of naltrexone, and, optionally, a binder. The composition may be compressed, cast, molded, or extruded to form a solid, compressed shape. In one embodiment, the compressed shape is a tablet, a pellet or a rod. As can be appreciated, a tablet is a disc-shaped object; a pellet is a spherical or ovoid shaped object, and a rod has a cylindrical, somewhat elongated shape. In an embodiment, the compressed shape has a density exceeding 1.00 g/cm³. In one embodiment, the pellets or rods are non-porous. In another embodiment, the composition is cast, molded, or compressed to form a tablet, pellet or rod with a density of between about 1.00 g/cm³-1.20 g/cm³.

The composition can comprise an optional binder with a melting point greater or equal to about 40° C. Examples include, but are not limited to, common tableting excipients, including lactose, maltose, mannitol, trehalose, hydroxypropylcellulose, polyvinylpyrrolidone, and polyethyleneglycol. Other binders include fatty (aliphatic) acids or fatty alcohols containing a minimum of 12 carbon atoms, dicarboxylic acids such as suberic or sebacic acid containing a minimum of 8 carbons, or any mixture thereof.

In one embodiment, the composition comprising the opioid antagonist includes one or more stabilizing excipients. Such excipients include metal chelating agents, such as a salt of ethylenediaminetetraacetic acid; a buffering agent, such as a phosphate salt, a carboxylate salt, or an amino acid; and antioxidants, such as ascorbic acid, an ascorbic acid ester, tocopherol, a tocopherol ester, cysteine, a cysteine ester or amide, methionine, a methionine ester or amide, butylated hydroxytoluene (BHT) or butylated hydroxyanisole (BHA).

The pellets or rods are fashioned such that the diameter of the pellet or rod is essentially equal to the inner diameter of the interior reservoir of the device. In one embodiment, the diameter of the pellets or rods is ≥95%, 96%, 97%, 98% or 99% of the inner diameter of the device interior reservoir. The composition can further comprise a lubricant.

The compressed shape is prepared and loaded into the device as a solid but is capable of dissolving in the presence of an aqueous fluid to produce an aqueous phase containing dissolved and/or suspended opioid antagonist, such as naltrexone, within the device's interior reservoir.

As noted above, the formulations described herein provide, when combined with an aqueous fluid, a mixture of aqueous and non-aqueous phases containing the opioid antagonist. When paired with a device, the aqueous phase formed within the device is in direct or indirect contact with a porous membrane. This permits the diffusion of dissolved opioid antagonist from the device interior into an environment of use. In one embodiment, this diffusion occurs over a sustained period of time, preferably of at least about two weeks to about twelve months, or at least about four weeks to six months.

In one embodiment, the delivery device is cylindrical or discoidal in shape, with dimensions less than or equal to about 8 mm in outer diameter and about 8 cm in length. In another embodiment, the length of the device is less than or equal to about 5 cm and the device outer diameter is less than or equal to about 5 mm. The walls of the device reservoir are made of a non-erodible and/or biocompatible material, such as titanium or surgical stainless steel, and one or both of the flat, circular, parallel ends of the device may comprise a porous partition or porous membrane. In an embodiment, the device is dimensioned to facilitate subcutaneous placement into a human subject; for instance, a cylindrical implant may be fashioned to be deposited within the subcutaneous space by means of a surgical trocar or implanter tool.

The porous membrane(s) or partition(s) mounted onto one or both ends of the device, for fluid communication with the device reservoir, are composed of a biocompatible material, such as polyvinylidene difluoride, sintered titanium or stainless steel, or a ceramic, such as alumina, titania, or silica. In a preferred embodiment, the total diffusive membrane surface area per device is between about 1 mm² and 50 mm². In a preferred embodiment, the porous partition has porosity of greater than or equal to about 30%, 35%, 40%, 45%, 50%, 55%, 60%, 65%, 70%, 75%, 80% or 85%, and a pore size of between about 0.05-0.75 microns, between about 0.1 to 0.45 microns, or between about 0.1 to 0.30 microns.

The interior volume of the implantable device is sufficient to hold a quantity of opioid antagonist formulation sufficient to dose a human subject for the intended therapeutic period. The therapeutic period may range from about 1 to 12 months, and preferably, is greater than about 2 months, 3 months, 4 months or 6 months and, optionally, less than about 14 months or about 12 months. In a preferred embodiment, the device has an interior volume of between about 500 μL and 1000 μL.

The interior diameter of the implantable drug delivery device is constant throughout its length, and, in one embodiment, the diameter of the interior reservoir is not more than 5% greater than the diameter of the solid, compressed shape (e.g., tablets, pellets or rods) that is/are placed into the interior reservoir. In other embodiments, the diameter of the interior reservoir is 5%, 4.4%, 4%, 3.5%, 3%, 2.5%, 2%, 1.5%, 1% or 0.5% greater than the diameter of the solid, compressed shape(s) to be placed into the interior reservoir. This configuration permits only a small amount of fluid to penetrate between the wall of the interior reservoir and the curved, outer surface of the compressed shape. A limited fluid volume limits the number of diffusion paths (and hence, the absolute diffusion rate) of particles exiting from the curved surface of the compressed shape relative to particles exiting from the orthogonal flat end of the compressed shape that is aligned with the porous membrane.

The compressed shape within the device may be hydrated in situ upon subcutaneous implantation of the device, or more preferably, the compressed shape can be hydrated immediately prior to subcutaneous implantation by a clinician introducing a liquid (e.g., a physiological buffer, isotonic saline, phosphate buffered saline, or aqueous propylene glycol) to the reservoir. The reservoir may be prepared and sealed under vacuum within a secondary container (for instance, a lyophilization vial sealed with a soft rubber septum) to facilitate this process. The liquid can be provided as part of a kit comprising a vial bearing the drug delivery device and a second vial bearing the hydration liquid.

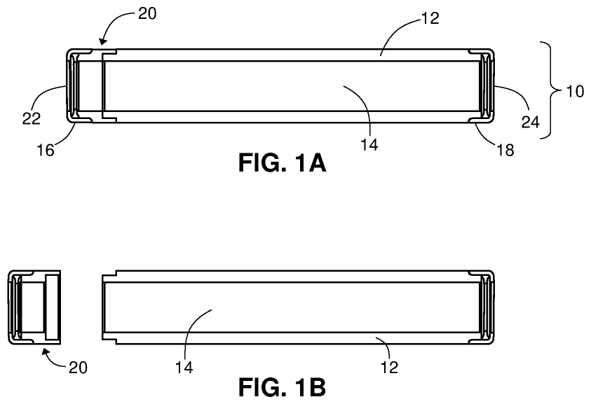

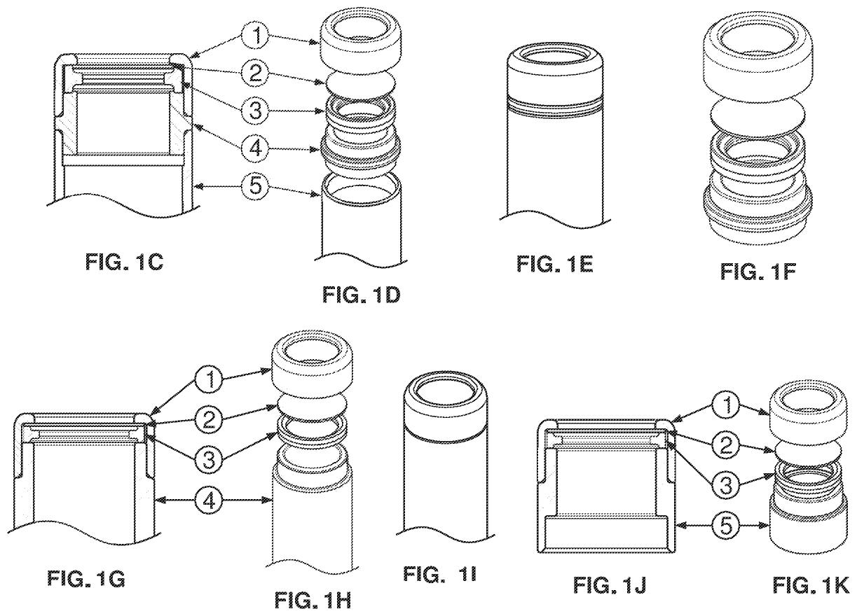

An example of a drug delivery device is provided in FIGS. 1A-1B. FIG. 1A illustrates a device 10, assembled and ready for implantation, in an anatomical compartment of a subject, such as under the skin or in the peritoneal cavity. The device is comprised of a non-erodible housing member 12 that defines an internal compartment or reservoir 14. Contained within the reservoir is a composition or formulation as described herein. Housing member 12 has first and second ends, 16, 18. First end 16 is sealed with a fluid-tight end-cap 20, seen best in FIG. 1B, that illustrates device 10 in its unassembled form. End cap 20 may optionally comprise a porous membrane or semi-permeable membrane or porous partition 22. Second end 18 is fitted with a porous membrane, semi-permeable membrane, or porous partition 24.

FIGS. 1C-1K illustrate the end caps and end cap subassembly portions of the exemplary drug delivery devices. The numbered elements of the subassembly illustrated in FIGS. 1C-1F are 1=cap, 2=porous membrane, 3=seal, 4=retention ring, and 5=drug device reservoir. The numbered elements of the subassembly illustrated in FIGS. 1G-1K are 1=cap, 2=porous membrane, 3=seal, 4=drug delivery device reservoir, and 5=retention ring.

The device interior contains, in one embodiment, a solid formulation containing an opioid antagonist, such as naltrexone base or a pharmaceutically acceptable salt thereof, and (optionally) a binder, such as lactose, maltose, hydroxypropylcellulose, polyvinylpyrrolidone, polyethyleneglycol, a fatty acid or fatty alcohol containing a minimum of 12 carbon atoms, a dicarboxylic acid containing a minimum of 8 carbons, or any mixture thereof, such that the melting point of the formulation is greater or equal to 40° C. The solid is compressed into macroscopically non-porous shapes, such as a tablet, a rod or a pellet, with, in one embodiment, a diameter that is at least about 95% of the interior diameter of the device. The tablets, rods or pellets have a density of greater than or equal to 1.00 g/cm³, or of between about 1.00 g/cm³ to 1.20 g/cm³, in one embodiment. The formulation may optionally comprise a lubricant, such as stearic acid, magnesium stearate, stearin, calcium stearate, sodium lauryl sulfate, glyceryl behenate, sodium benzoate, talc, and the like.

As used herein, the terms “porous membrane” and “porous partition” intend a structural member that has a plurality of pores in the nanometer or micrometer (μm) range, preferably in the 0.1-0.45 μm range. The drug elutes from the device interior by diffusing across the partition; it permits dissolved naltrexone (or a similar opioid antagonist such as naloxone) to exit the device but retains the undissolved drug composition within a solid state. In order to deliver a therapeutic dose from the device of the present invention, the total surface area of the porous partitions incorporated into each device is 1 mm² to 50 mm². The drug delivery device is described in U.S. 2011/0106006, which is incorporated by reference herein. In a preferred embodiment, the membrane/partition has a porosity of greater than or equal to 70%. In another embodiment, the membrane/partition has a porosity of greater than or equal to 50%.

Studies were conducted to evaluate the release rate and kinetic order of release from drug delivery devices containing a solid formulation of an opioid antagonist, using naltrexone base as an example. As described in Example 1, two sets of cylindrical drug delivery devices were prepared. For Set 1: inner diameter (ID), 4.3 mm; outer diameter (OD), 5.00 mm; internal volume, 474 μL; length, 40 mm. For Set 2: ID, 4.5 mm; OD, 5.00 mm; internal volume, 650 μL; length, 42.2 mm All device reservoirs and caps were fabricated from titanium, and the ends of all devices were capped with circular 0.1 μm polyvinylidene difluoride membranes (DURAPORE™, Millipore, Inc.). The total diffusive area per device was calculated to be approximately 19.4 mm² for the first set, and 21.6 mm² for the second set. Devices in the first set were loaded with approximately 325 mg of naltrexone base as a fine powder. Devices in the second set were loaded with approximately 570 mg of tablets, consisting of 93% naltrexone base, 5% polyvinylpyrrolidone (40 kD), and 2% stearic acid as a lubricant, by mass. Tablets were prepared by grinding the individual powders together, followed by tableting on a single-punch tablet press equipped with a custom 4.35 mm diameter die set. Compression parameters were adjusted to yield a tablet density of approximately 1.09 g/cm³. In one embodiment, the tablet has a density of at least about 0.75 g/cm³, at least about 0.80 g/cm³, at least about 0.85 g/cm³, at least about 0.90 g/cm³, at least about 0.95 g/cm³, at least about 1.0 g/cm³, at least about 1.05 g/cm³, at least about 1.10 g/cm³, or at least about 1.15 g/cm³.

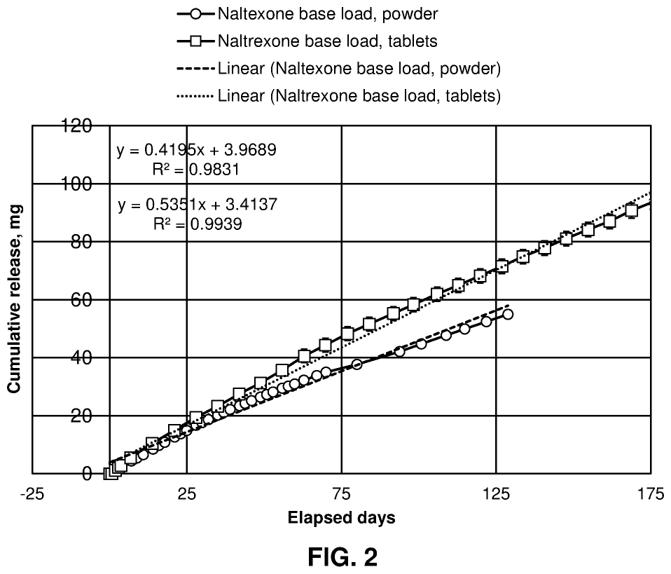

The drug delivery devices were sealed in lyophilization vials under vacuum and hydrated with 0.9% saline immediately prior to testing in vitro or in vivo. FIG. 2 shows the results of the in vitro testing, where the cumulative release of naltrexone (mg) from each device set is plotted as a function of time (days). Data from drug delivery devices filled with the naltrexone powder composition are shown in blue circles; data from devices filled with the tableted naltrexone composition are shown in orange squares. Following hydration at t−0, devices (n=3) were incubated at 37° C. in jars containing phosphate buffered saline as a receiving buffer. Buffer exchanges were performed periodically for each set, and the drug content of retained buffer samples were quantified by reverse-phase HPLC. As seen in FIG. 2, over a period of approximately 6 months, the compressed, solid composition comprising naltrexone provided for naltrexone elution from the devices at a constant rate (r2=0.994) at an average rate of 0.5-0.6 mg/day.

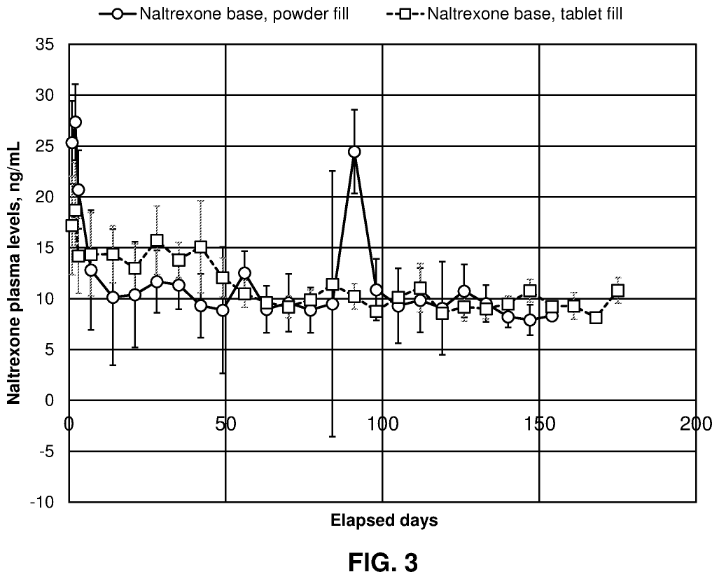

FIG. 3 shows plasma concentrations of naltrexone (ng/mL) plotted as a function of time (days) in rats implanted with devices loaded with either finely powdered naltrexone base (n=5) or a tableted naltrexone composition (n=3). Plasma samples were periodically obtained from these animals, frozen, and later analyzed using a LC/MS method to quantify plasma levels of naltrexone. Data was then normalized to the initial weight of each animal to account for weight gain over the course of the study. Plasma levels were found to remain relatively constant between 10 ng/mL and 15 ng/mL for at least 5 months.

It is contemplated that a drug delivery device such as one described herein can be implanted at any suitable implantation site using methods and devices well known in the art. As noted below, an “implantation site” is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are preferred because of convenience in implantation and removal of the drug delivery device. Exemplary subcutaneous delivery sites include under the skin of the abdomen, arm, shoulder, neck, back, or leg. Sites within a body cavity are also suitable implantation sites. Methods for implanting or otherwise positioning drug delivery devices for subcutaneous delivery of a drug are well known in the art. In general, placement of the drug delivery device will be accomplished using methods and tools that are well known in the art and performed under aseptic conditions with at least some local or general anesthesia administered to the subject.

### Methods of Treatment

In other aspects, methods of treatment using the compositions and devices described herein are contemplated. In one embodiment, a method for sustained, controlled delivery of an opioid antagonist is contemplated, where a device paired with a composition as described herein is provided.

In one embodiment, the composition is a dry formulation, such as a powder, tablet, pellet or rod. The dry formulation is placed within the interior reservoir of the device. A porous partition or membrane is secured to the device, and, in one embodiment, secured to one or both ends of the device, where the interior reservoir of the device is in fluid communication with an environment external to the device via the porous partition/membrane. If desired, the device can be placed into a glass vial and sealed under vacuum at pressures ranging from 0.02 Torr to 100 Torr. The vialed devices are packaged, distributed and stored in dry form. In one embodiment, prior to implanting the device, an aqueous medium, such as 0.9% saline or water for injection, is introduced into the vial. The vacuum draws the aqueous fluid into the vial and into the void space within the device reservoir (i.e., the interior reservoir volume not occupied by the solid, dry formulation). The fluid wets the membrane and formulation and creates a diffusion path for dissolved drug; diffusion of the drug through the membrane ensues immediately following hydration. Undissolved drug is retained in the device interior reservoir. However, as dissolved drug diffuses from the device, undissolved drug enters solution and becomes available for diffusion across the partition.

A preferred device implantation site is the subcutaneous tissue in the abdomen or the arm. Implantation is accomplished, for example, with an implanter tool and other instruments provided in a surgical kit. Following implantation, the device(s) elutes sufficient drug in vivo to achieve a plasma concentration sufficient for therapy for a period of at least about 1 month and up to 3 months, 4 months, 6 months or 12 months. For example, when the opioid antagonist is naltrexone base, the target plasma concentration is between about 0.01-0.50 ng/ml for at least 1 month and up to 12 months, as this level of exposure is projected to correspond to the average plasma levels obtained from oral naltrexone dosed at approximately 1/10^{th} the level used to treat opioid use disorder. In another example, when the opioid antagonist is naltrexone base, the target plasma concentration is at least about 0.01 ng/ml, 0.02 ng/mL, 0.03 ng/mL, 0.04 ng/mL, 0.05 ng/mL, 0.06 ng/ml, 0.07 ng/mL, 0.08 ng/mL, 0.09 ng/ml or 0.1 ng/mL and less than 0.50 ng/ml for at least 1 month and up to 12 months, as this level of exposure is projected to correspond to the average plasma levels obtained from oral naltrexone dosed at approximately 1/10^{th} the level used to treat opioid use disorder. In other embodiments, the device comprises an amount of opioid antagonist to provide a therapeutic blood (plasma or serum or blood) concentration for between 1-36 months, 1-24 months, 1-12 months, 1-8 months, 1-6 months, 1-4 months, 1-3 months, 2-36 months, 2-24 months, 2-12 months, 2-10 months, 2-8 months, 2-6 months, 2-4 months, 3-36 months, 3-24 months, 3-12 months, 3-10 months, 3-9 months, 3-8 months, 3-6 months, or 3-4 months. Sufficient output of opioid antagonist from the device can be achieved, for example, by balancing the shape and size of the device, mass of drug loaded and the porosity and diffusive surface area of the porous partition and number of implanted devices.

In one embodiment, a device with an internal volume of between about 500-1000 μL comprises a composition of naltrexone or a pharmaceutically acceptable salt thereof. The amount of opioid antagonist within the device is sufficient to provide a therapeutically relevant plasma level of naltrexone and its active metabolite, 6-β-naltrexol, for a period of at least about 1 month, at least about 2 months, at least about 3 months, or for between 1-36 months, 1-24 months, 1-18 months, 1-12 months, 1-6 months, 1-3 months, or 2-36 months, 2-24 months, 2-28 months, 2-12 months, 2-6 months or 2-3 months. In another embodiment, the amount of opioid antagonist within the device is sufficient to provide a therapeutically relevant plasma level of naltrexone exclusive of its active metabolite, 6-β-naltrexol, for a period of at least about 1 month, at least about 2 months, at least about 3 months, or for between 1-36 months, 1-24 months, 1-18 months, 1-12 months, 1-6 months, 1-3 months, or 2-36 months, 2-24 months, 2-28 months, 2-12 months, 2-6 months or 2-3 months.

In another embodiment, a method for maintaining therapeutic plasma levels of naltrexone, naloxone, or a similar opioid receptor antagonist is contemplated, thus reducing the frequency or intensity of symptoms associated with a chronic inflammatory, neuroinflammatory, or metabolic disorder over a period of time.

### EXAMPLES

The following examples are illustrative in nature and are in no way intended to be limiting.

### Example 1

### Dissolution-Based Device for the Delivery of Naltrexone at a Constant Rate—In Vitro Evaluation

Tablets comprised of naltrexone base (93% by mass), polyvinylpyrrolidone (40 kD average molecular weight; 5% by mass) and, as a lubricant, stearic acid (2% by mass) were prepared by briefly grinding the powders together, followed by tableting on a single-punch tablet press (Vanguard model CP-501; Spring, TX) equipped with a custom 4.35 mm diameter die set. Compression parameters were adjusted to yield a tablet density of approximately 1.09 g/cm³. These were used to fill cylindrical drug delivery devices (4.50 mm ID, 5.00 OD, 42.2 mm length, 650 μL internal volume) fashioned from titanium and capped with two circular, parallel 0.1 micron polyvinylidene difluoride (DURAPORE®) membranes (21.6 mm² total diffusive area per device). All devices were vacuum back-filled with 0.9% saline and transferred to jars containing phosphate-buffered saline at a volume of ˜100 mL. The sealed jars were then incubated at 37° C. The receiving buffer was periodically exchanged (minimum once per week), and small aliquots (˜500 μL) of retained fluid were analyzed by high pressure liquid chromatography (HPLC) to quantify the drug. Release of naltrexone from this device configuration is shown in FIG. 2 (orange square symbols).

The in vitro release profile of a powder formulation of naltrexone base was also measured. Devices were fashioned from titanium with the following dimensions: ID, 4.3 mm; OD, 5.00 mm; internal volume, 474 μL; length, 40 mm. Each device reservoir was loaded with approximately 325 mg of naltrexone base as a fine powder, before being capped with two circular, parallel 0.1 micron polyvinylidene difluoride (DURAPORE®) membranes (19.4 mm² total diffusive area per device). The in vitro release rate was measured as above. Release of naltrexone from this device configuration is also shown in FIG. 2 (blue circular symbols).

### Example 2

### Dissolution-Based Device for the Delivery of Naltrexone at a Constant Rate—In Vivo Evaluation

Drug delivery devices were prepared as described for Example 1. Tablets comprised of naltrexone base (93% by mass), polyvinylpyrrolidone (40 kD average molecular weight; 5% by mass) and stearic acid (2% by mass) were prepared by briefly grinding the powders together, followed by tableting on a single-punch tablet press (Vanguard model CP-501; Spring, TX) equipped with a custom 4.35 mm diameter die set. Compression parameters were adjusted to yield a tablet density of approximately 1.09 g/cm³. These were used to fill cylindrical drug delivery devices (4.50 mm ID, 5.00 OD, 42.2 mm length, 650 μL internal volume) capped with two circular, parallel 0.1 micron polyvinylidene fluoride (DURAPORE®) membranes (21.6 mm² total diffusive area per device). The average fill weight was approximately 570 mg of formulation per device. These devices were sealed in lyophilization vials under vacuum and terminally sterilized. Devices were vacuum back-filled with 0.9% saline and implanted singly into rats (n=3). Plasma samples were periodically obtained from these animals, frozen, and later analyzed using a LC/MS method to quantify plasma levels of naltrexone over time. Data was then normalized to the initial weight of each animal to account for weight gain over the course of the study. Plasma levels were found to remain relatively constant between 10 ng/mL and 15 ng/mL for over 5 months post-implantation (FIG. 3, orange square symbols).

Also as described in Example 1, devices were fashioned from titanium with the following dimensions: ID, 4.3 mm; OD, 5.00 mm; internal volume, 474 μL; length, 40 mm. Each device reservoir was loaded with approximately 325 mg of naltrexone base as a fine powder, before being capped with two circular, parallel 0.1 micron polyvinylidene difluoride (DURAPORE®) membranes (19.4 mm² total diffusive area per device). These devices were sealed in lyophilization vials under vacuum and terminally sterilized. Devices were vacuum back-filled with 0.9% saline and implanted singly into rats (n=3). Plasma samples were periodically obtained from these animals, frozen, and later analyzed using a LC/MS method to quantify plasma levels of naltrexone over time. Data was then normalized to the initial weight of each animal to account for weight gain over the course of the study. Plasma levels were found to remain relatively constant between 10 ng/mL and 15 ng/mL for approximately 5 months post-implantation (FIG. 3, blue circular symbols).

1. A device for delivery of an opioid antagonist, comprising:
a device comprising an interior reservoir with an inner diameter, a first end, a second end, and a porous partition, the device dimensioned for subcutaneous implantation into a subject;
a solid formulation comprising an opioid antagonist and one or more of a binder, a stabilizing excipient, and a lubricant, the solid formulation in the form of a compressed shape with a diameter equal to or greater than 80% of the inner diameter of the interior reservoir,
wherein the compressed shape when hydrated forms an aqueous phase in the interior reservoir of the device, the aqueous phase comprising dissolved opioid antagonist that is released from the device by diffusion across the porous partition for a period of at least about one month.

2-4. (canceled)

5. The device of claim 1, wherein the compressed shape is selected from a tablet, a pellet and a rod.

6. The device of claim 5, wherein the compressed shape has a diameter equal to or greater than 90% of the inner diameter of the interior reservoir.

7. The device of claim 1, wherein the solid formulation comprises a binder.

8. The device of claim 7, where the binder is selected from the group consisting of lactose, maltose, mannitol, trehalose, hydroxypropylcellulose, polyvinylpyrrolidone, polyethyleneglycol, a fatty acid or alcohol containing a minimum of 12 carbon atoms, and a dicarboxylic acid containing a minimum of 8 carbon atoms.

9. (canceled)

10. The device of claim 9, wherein the solid formulation comprises a stabilizing excipient selected from a metal chelating agent, a buffering agent, and an antioxidant.

11. The device of claim 1, wherein the solid formulation has a melting point greater or equal to about 40° C.

12-20. (canceled)

21. The device of claim 1, wherein the opioid antagonist is naltrexone base or a pharmaceutically acceptable salt of naltrexone.

22-25. (canceled)

26. The device of claim 1, wherein the opioid antagonist is in amount to maintain a therapeutically effective plasma level of the opioid antagonist and/or a metabolite thereof for the period.

27-28. (canceled)

29. A method for in vivo delivery of an opioid antagonist, comprising:
providing a device according to claim 1, and
implanting the device into a subcutaneous space of a human subject, whereby said implanting results in an aqueous fluid in the subcutaneous space entering the device to hydrate the formulation, or
prior to implanting the device into a subcutaneous space of a human subject, contacting the device with an aqueous fluid to wet the device membranes and formulation.

30-32. (canceled)

33. A method for the treatment of an inflammatory, neuroinflammatory, or metabolic disorder, comprising:
providing a device according to claim 1,
administering the device to a human subject by subcutaneous implantation, whereby said administering provides delivery of the opioid antagonist in an amount that provides a therapeutic blood level for a period of between about 1-36 months.

34-38. (canceled)

39. The device of claim 10, wherein the metal chelating agent is ethylenediaminetetraacetic acid.

40. The device of claim 10, wherein the buffering agent is selected from the group consisting of a phosphate salt, a carboxylate salt, and an amino acid.

41. The device of claim 10, wherein the antioxidant is selected from ascorbic acid, an ascorbic acid ester, tocopherol, a tocopherol ester, cysteine, a cysteine ester or amide, methionine, a methionine ester or amide, butylated hydroxytoluene (BHT) and butylated hydroxyanisole (BHA).

42. The device of claim 1, wherein the solid formulation comprises a lubricant selected from the group consisting of stearic acid, magnesium stearate, stearin, calcium stearate, sodium lauryl sulfate, glyceryl behenate, sodium benzoate, and talc.
